(19) United States
(12) Patent Application Publication
(10) Pub. No.: US 2010/0042013 A1
(43) Pub. Date: Feb. 18, 2010

(54) SYSTEM AND APPARATUS FOR WIRELESS HIGH-FREQUENCY TEMPERATURE ACQUISITION AND ANALYSIS

(75) Inventors: DAVID CUESTA FRAU, ALCOI (ES); PEDRO MATEO RIOBO ABOY, BEAVERTON, OR (US); MANUEL VARELA ENTRECANALES, MADRID (ES)

Correspondence Address: ABOY&ASSOCIATES PC;www.aboypatentlaw.com, 522 SW 5th Ave, Suite 1265, Portland, OR, 97204, US

(73) Assignee: INNOVATEC SL, ALCOI - Alicante (ES)

(21) Appl. No.: 12/541,914

(22) Filed: Aug. 15, 2009

Related U.S. Application Data
(60) Provisional application No. 61/089,545, filed on Aug. 17, 2008.

(51) Int. Cl. A61B 5/00; G06F 15/00; G01K 1/00

(52) U.S. Cl. 600/549; 702/130

(57) ABSTRACT

We disclose a system, an apparatus, and a method for high frequency temperature monitoring and analysis. According to a disclosed embodiment the system comprises: (a) a wireless temperature acquisition and logging device especially designed for multi-day, high-frequency, and high-resolution temperature sampling; and (b) an analysis system implemented in a digital computer with one or more processors in order to analyze and characterize said temperature using a plurality of methods including complexity analysis techniques such as Lempel-Ziv complexity, Approximate Entropy, Sample Entropy, Multiscale Entropy, and Detrended Fluctuation Analysis; and other statistical time-series analysis techniques. According to one embodiment the temperature monitoring system is designed to capture the dynamic aspects of temperature in order to enable researchers and clinicians to study temperature regulation, thermal physiology, and clinical thermometry.

### CROSS REFERENCE TO RELATED APPLICATIONS

This application claims the benefit of U.S. Provisional Application No. 61/089,545 filed on 2008-08-17 by the present inventors, which is incorporated herein by reference.

### BACKGROUND FIELD OF INVENTION

This invention relates to clinical monitoring systems. Specifically, it relates to portable temperature monitoring systems.

### BACKGROUND RELATED ART

Temperature is a continuous variable that can be measured and analyzed quantitatively. However, in clinical practice it is primarily used as an intermittent qualitative variable with often a poorly defined cut-off point. Despite its potential as a noninvasive physiologic signal to continuously assess patient status, its clinical use is often limited to a simple binary decision. While research studies have shown the usefulness of the temperature time-series for patient assessment and its prognostic value has been documented, the use of high-frequency/high-accuracy temperature time-series and associated analysis metrics have not been extended to clinical environments due to the lack of commercial temperature monitoring systems with adequate sampling frequency, resolution, and analysis capabilities. Consequently, even though thermal physiology has undergone big conceptual and technical changes, clinical thermometry has remained a rather stagnant discipline, and its conceptual framework has remained static for the last decades due to the lack of adequate monitoring systems.

Recent work involving analysis of high-accuracy and high-frequency temperature recordings has shown the importance of analyzing temperature as a dynamic time-series as opposed to a simple constant only useful to make a simple binary decision (febrile vs. nonfebrile). These important findings and clinical applications require medical devices capable of monitoring high-resolution temperature (e.g. at a rate of 1 sample per minute) during a period of several days. For instance, analysis metrics derived from the temperature time-series have been shown to correlate with the clinical status in critically ill patients. Specifically, analysis metrics derived from the temperature time-series have been shown to serve as a good indicator of poor prognosis in patients with multiple organ failure. For instance, studies have shown that the predictive ability of the metrics derived from the temperature time-series alone were comparable to the Sequential Organ Failure Assessment (SOFA) score; and that analysis of the temperature-time series provides clinically useful information for noninvasive and real-time status assessment, since the metrics derived from the temperature time-series have been found to correlate with patient survival.

Recent work has shown the importance of analyzing temperature as a dynamic time-series as opposed to a simple constant only useful to make a simple binary decision (febrile vs. nonfebrile). These important findings and clinical applications require medical devices capable of monitoring high-resolution temperature at a high sampling rate during a period of several days. Currently there are no high-accuracy, high-frequency temperature monitoring apparatus available intended to serve as a clinical device to enable physicians to implement current advances in thermal physiology as part of their day-to-day clinical practice.

### SUMMARY

According to one disclosed embodiment, and without limitation, the system for high frequency temperature monitoring and analysis comprises: (a) a wireless temperature acquisition and logging device especially designed for multi-day, high-frequency, and high-resolution temperature measurement; and (b) an analysis application implemented in a digital computer with one or more processors in order to analyze and characterize said temperature using a plurality of methods including complexity analysis techniques such as Lempel-Ziv complexity, Approximate Entropy, Sample Entropy, Multiscale Entropy, and Detrended Fluctuation Analysis; and other statistical time-series analysis techniques.

According to one embodiment the temperature monitoring system is designed to capture the dynamic aspects of temperature in order to enable researchers to conduct further studies in the area of temperature regulation, thermal physiology, and clinical thermometry. Additionally, it is intended to serve as a clinical device to enable physicians to dynamically assess the physiological status of a patient based on complexity analysis of the temperature time-series.

### BRIEF DESCRIPTION OF THE DRAWINGS

Disclosed embodiments are illustrated by way of example, and not by way of limitation, in the figures of the accompanying drawings.

FIG. 1 shows a block diagram of the system according to one embodiment.

FIG. 2 shows the temperature device according to one embodiment.

FIG. 3 shows a block diagram of the temperature device according to one embodiment.

FIG. 4 shows a schematic of the thermistor amplifier circuit according to one embodiment.

FIG. 5 shows a schematic of the device power source according to one embodiment.

FIG. 6 shows a schematic of the voltage references according to one embodiment.

FIG. 7 shows a screenshot of the analysis system illustrating a high-frequency, high-accuracy temperature time series.

### DETAILED DESCRIPTION

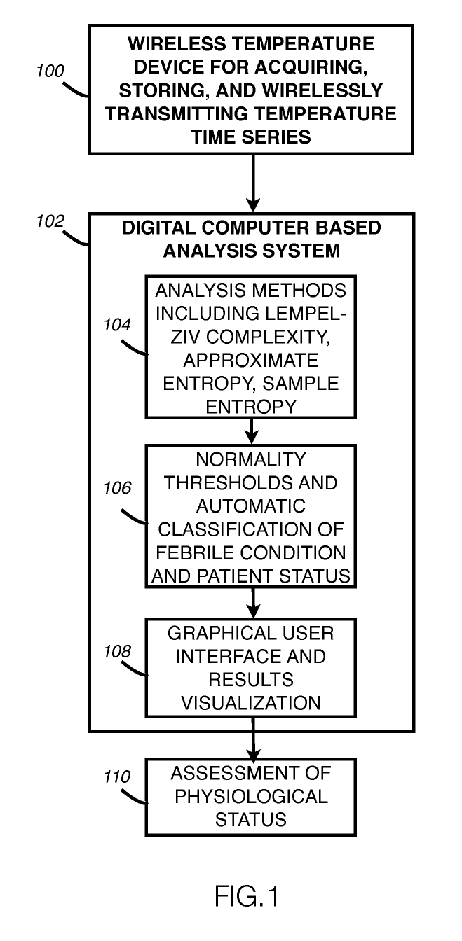

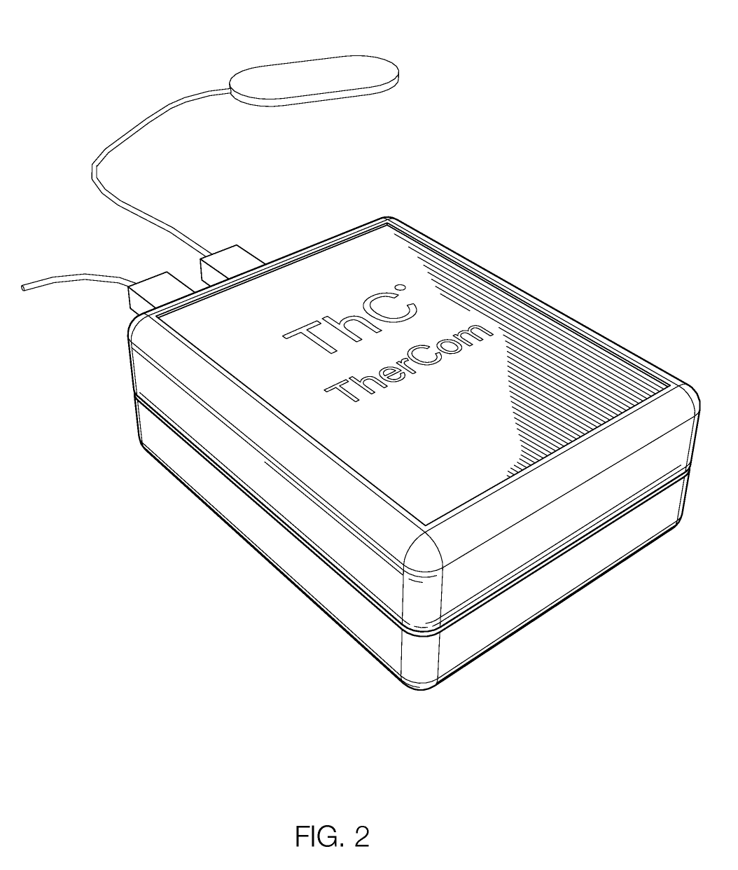

FIG. 1 shows a block diagram of the system according to one embodiment. According to an embodiment, the system for taking temperature measurements and analyzing said measurements comprises: (a) a wireless temperature acquisition and logging device 100 for acquiring, storing, and wirelessly transmitting said temperature measurements; and (b) an analysis system 102 implemented in a digital computer with one or more processors in order to analyze and characterize said temperature using one or more time-series analysis methods. FIG. 2 shows the temperature device 100 according to one embodiment.

According to one disclosed embodiment, and without limitation, the system for high frequency temperature monitoring and analysis comprises: (a) a wireless temperature acquisition and logging device 100 especially designed for multi-day, high-frequency, and high-resolution temperature measurement; and (b) an analysis system 102 implemented in a digital computer with one or more processors in order to analyze and characterize said temperature using a plurality of methods including complexity analysis techniques such as Lempel-Ziv complexity, Approximate Entropy, Sample Entropy, Multiscale Entropy, and Detrended Fluctuation Analysis; and other statistical time-series analysis techniques 104. In this application area high-frequency sampling is considered temperature sampling (measurements) at a rate equal or above 1 temperature measurement per minute.

According to one embodiment, a system for taking temperature measurements, analyzing said measurements, and characterizing fever and febrile conditions to help make clinical decisions regarding a patient's condition is disclosed. This system comprises: (a) a wireless temperature acquisition and logging device 100 especially designed for multi-day, high-frequency, and high-resolution temperature measurements for acquiring, storing, and wirelessly transmitting said temperature measurements; and (b) an analysis system 102 implemented in a digital computer with one or more processors in order to analyze and characterize said temperature using one or more complexity analysis methods such as Lempel-Ziv complexity, Approximate Entropy, Sample Entropy, Multiscale Entropy, and Detrended Fluctuation Analysis 104, and generate an output temperature complexity metric, said output temperature complexity metric is automatically compared against one or more normality thresholds 106 obtained from population studies to characterize specific conditions or risks to help make said clinical decisions regarding said patient's condition (physiological status) 110 which is visualized as part of the graphical user interface (GUI) 108. The system provides a tool to assess the physiological status of patients by conducting complexity analysis of the temperature time-series. This time-series can be monitored and analyzed to assess the thermoregulatory mechanism and clinical status of patients across all temperatures (febrile, normal temperature, hypothermic, etc.), to assess the circadian rhythms, and to help with the assessment in a plurality of pathologies. For instance, the system can be to used study thermoregulatory mechanisms (without fever) in certain diseases such as autonomic neuropathy and for assessing the temperature in hypothermic patients (hypothermia accidental or induced hypothermia).

According to one embodiment, a method for assessing a patient's condition using the system described herein is disclosed. The method comprises the following method steps: (a) collecting a temperature time-series using a temperature acquisition and logging device especially designed for multi-day, high-frequency, and high-resolution temperature measurements; (b) automatically calculating and displaying a complexity metric at user-specified time intervals and the complexity metric is calculated using a user-specified window length; and (c) assessing said patient's condition by comparing said complexity metric against one or more normality thresholds known to be indicative of a particular clinical condition. Complexity metrics include Lempel-Ziv complexity, Approximate Entropy, Sample Entropy, Multiscale Entropy, and Detrended Fluctuation Analysis 104. The complexity metric can be updated for every temperature sample (i.e. overlap of 99%) using any user-specified window length (e.g. from 5 seconds to hours). The normality thresholds and confidence intervals are determined by conducting clinical studies to characterize the temperature time-series dynamics associated with specific conditions, pathologies, and risks.

### A. Uses of a Disclosed Embodiment.

According to one embodiment the temperature monitoring system is designed to capture the dynamic aspects of temperature in order to enable researchers to conduct further studies in the area of temperature regulation, thermal physiology, and clinical thermometry. Additionally, it is intended to serve as a clinical device to enable physicians to dynamically assess the physiological status of a patient based on complexity analysis of the temperature time-series and to assess/classify the temperature condition.

Embodiments can be designed to serve as systems and apparatus to address common problems encountered in the clinical practice and research involving temperature monitoring and analysis. These include:
Monitoring temperature in admitted patients. Temperature is normally measured every 8-12 hours. Common knowledge assumes that if the patient develops a fever, either it will persist until the next lecture, or the patient will be so sick as to call the nurse, and the fever will be detected. Nevertheless, there are no empirical studies to back up this practice. A continuous monitoring of the patient's temperature can help detect intermittent fever bouts and unleash the consequent diagnostic and therapeutic interventions more objectively 106.
Detecting fever in an outpatient setting. A common complaint in an outpatient clinic is the presence of fever. Fever often develops in the evening, and may not be confirmed during the visit to the clinic. Having the capability to record for a 24 or 48 hour period (“temperature Holter monitoring”) eliminates this problem and enables clinicians to arrive to more precise diagnosis 106.
Detecting bacteremia as early as possible, eventually before the fever spike. The presence of bacteria in blood triggers the release of a number of cytokines that stimulate certain hypothalamic nuclei and start several physiologic measures aiming to rise the core temperature. The first of these mechanisms is a vasoconstriction of peripheral vessels, aiming to bypass the blood from the subcutaneous tissue to the deep tissues and therefore minimizing heat dissipation. This is reflected in an abrupt fall in peripheral temperature. Measuring simultaneously central and peripheral temperature has the potential to provide early signs of the development of bacteremia prior to the appearance of a full-blown fever. Furthermore, blood cultures would arguably be more sensitive in this setting than once the fever mechanisms have been unleashed.
Providing new ways of categorizing fever. Classic fever patterns (i.e. “hectic”, “recurrent”, etc) are limited. New analytical techniques derived from complexity time-series analysis (Approximate Entropy (ApEn), Sample Entropy, Lempel Ziv algorithm, Detrended Fluctuation analysis) can be used to study and classify temperature, even in the absence of fever. For instance, there is a good correlation between physiological status and ApEn of the temperature profile in critically ill patients, and that ApEn is a prognostic marker as good as conventional scoring systems, with the advantage of being non-invasive and a continuous, rather than episodic, evaluation. A temperature monitoring system that comprises an apparatus to measure temperature with high frequency and complexity analysis techniques could be used to characterize fever and febrile conditions 106.

### B. Implementation of the Temperature Monitoring Device According to an Embodiment.

The following section discloses an embodiment of the temperature monitoring device 100. This embodiment is meant simply to illustrate how to construct such a device and should not be interpreted as limiting. The description details sample voltages, components, specifications, component values, etc. in order to enable those skilled in the art to construct a working embodiment without undue experimentation. While particular embodiments are described, it is understood that after learning the teachings included in this description, modifications and generalizations will be apparent to those skilled in the art without departing from the spirit of the disclosed embodiment. For instance, different voltages, components, specifications, component values, etc can be used without departing from the spirit of the disclosed embodiment.

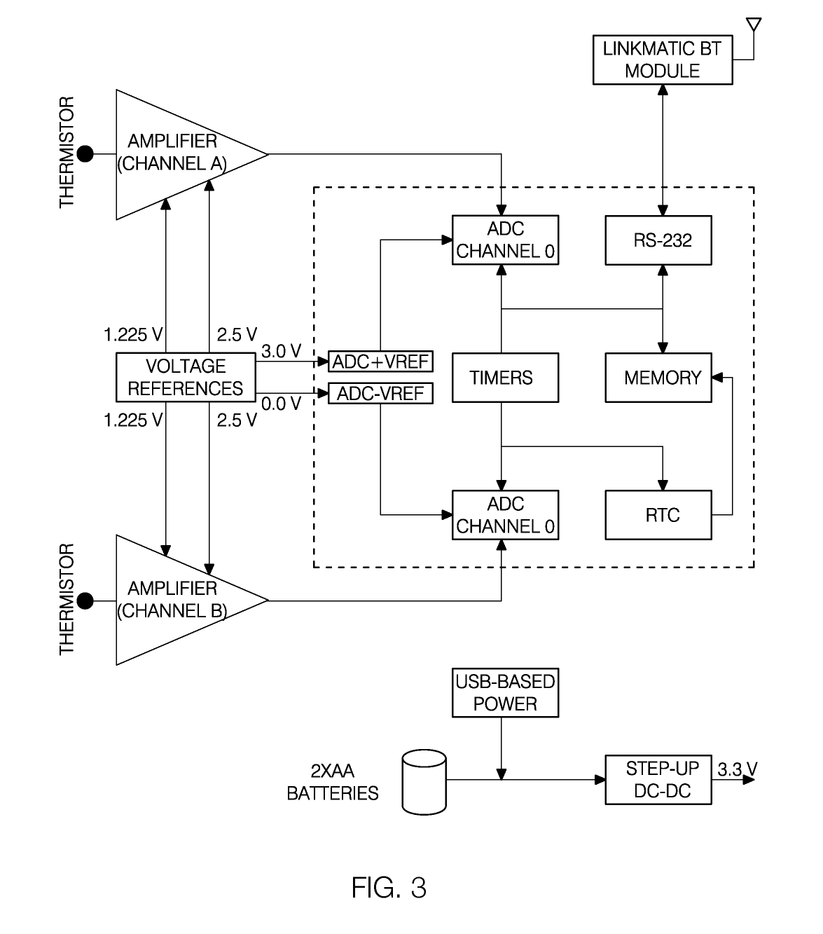

FIG. 3 shows a block diagram of one embodiment of the temperature device 100 and how the components are interrelated. This specific embodiment of the temperature monitor includes temperature sensors, a signal conditioner and amplifier for each channel, an analog to digital converter, a microcontroller, flash memory, and a Bluetooth module. It also includes a DC-DC converter to power the device from two standard AA batteries. Other embodiments use a different battery source. Samples are transmitted via Bluetooth connection to the host computer at a rate of one sample per minute in real time mode, or off-line in data logger mode. At the host computer, data can be visualized and processed. The software tool includes a number of menu options for signal editing, entropy calculations, time-series analysis, and device configuration.

According to one embodiment, YSI400 thermistors are used as the temperature sensors. The accuracy of the sensors is ±0.02° C. and the resolution is 0.01° C. Thermistor readings are converted using the standard YSI400 curve and the Steinhart-Hart polynomial which provides an accuracy far better than that of the sensors or parts themselves, ±0.0025° C. at 25° C. with a resolution of 0.0001° C. To calibrate the device, individual ultra high precision resistors (0.01% tolerance, ±2 ppm/° C.) are connected in place of the thermistors. The gain and offset are simultaneously adjusted by means of linear interpolation between reference points obtained.

As shown in FIG. 3 according to one embodiment the sensor resistance is turned into voltage by means of a precision current source, and this voltage then enters the amplifier to get an output range from 0V to 3.0V. The temperature measurements range from 19.00° C. to 41.00° C. Self heating is avoided by powering the sensors during a very short period of time. Sensors can be attached to the skin of the patient for peripheral temperature acquisition, or be embedded in an orifice (usually the ear canal) for central temperature monitoring.

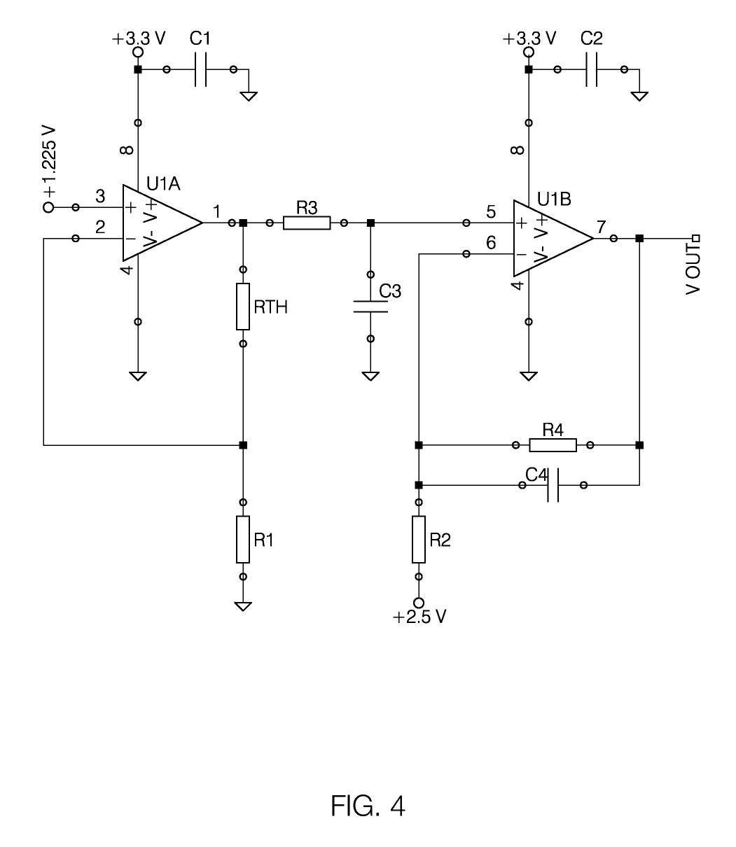

According to one embodiment, to obtain the temperature from a thermistor it is necessary to measure its resistance by quantifying the voltage across it. This is carried out by the thermistor amplifier shown in FIG. 4. The first stage of this circuit is a precision current source. As current intensity varies along with the thermistor resistance, it has to be kept constant. This is accomplished by U_{1A} configured as a current source. Current delivered to the thermistor Rₜₕ is given by the input voltage and R₁. To ensure high accuracy, the input voltage is obtained from a precision reference, and R₁ must be a low tolerance resistor (0.1%). Since the minimum thermistor resistance is 1.152Ω at 41.00° C., and the maximum is 1.739Ω at 31.00° C., an input reference voltage of 1.225 V and R₁=1K58 is chosen. Thus, the current flowing through Rₜₕ is constant and equals 77.5 mA.

According to one embodiment, the output Rₜₕ voltage is low-pass filtered by R₃ and C₃. The values chosen for these parts are 4K7 and 22 μF, respectively, in order to get a cut-off frequency of 1.5 Hz. Finally, the signal is amplified and further low-pass filtered by U_{1B}. An output offset of 2.5V is added to obtain a final output voltage range from 0 to 3.0V, using the gain set by R₄=5K49 and R₂=K02. C₁ and C₂ are decoupling capacitors to prevent noise from the power supply from entering the signal. V_{OUT} is the input to the ADC.

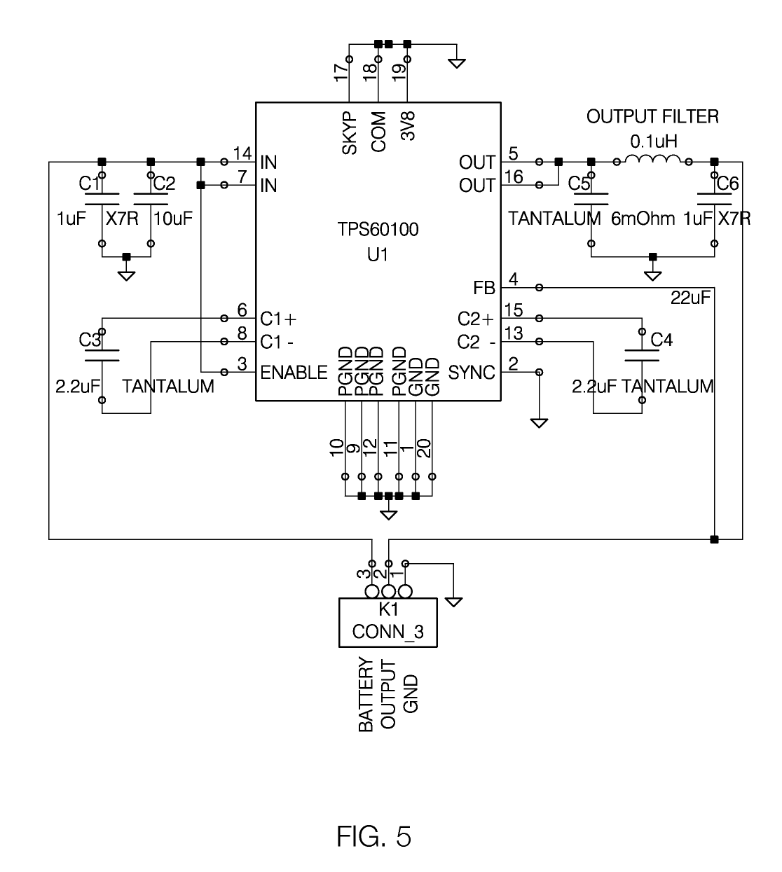

According to one embodiment, two NiMH batteries are used in series to provide an output voltage of approximately 2.5V. Since the temperature apparatus parts require a higher and stable voltage of 3.3V, a regulator is needed to boost and keep the voltage constant. The circuit for this task is shown in FIG. 5.

According to one embodiment a step-up charge pump such as the TPS60100 is used. It generates 3.3V±4% from the battery input, and provides a maximum output current of 200 mA. This is sufficient to power the whole device (maximum current consumption is 60 mA during Bluetooth operation) with an efficiency up to 90%. This dc-to-dc converter does not require any external inductor, only four capacitors. Ceramic capacitors are recommended for their low equivalent series resistance, that enables them to be charged and discharged very fast and therefore filter out spikes. An additional output filter (FIG. 5) is included to further reduce the ripple. Low power consumption is also important in portable devices. The proposed embodiment minimizes the power consumption by:
Switching off external circuits when not needed. I/O pins are used to power the thermistor amplifier and the voltage references.
The microcontroller periodically goes to sleep and the watchdog timer awakes it for temperature sampling.
Clocking the microcontroller at a lower frequency.
Configuring unused pin ports as outputs.

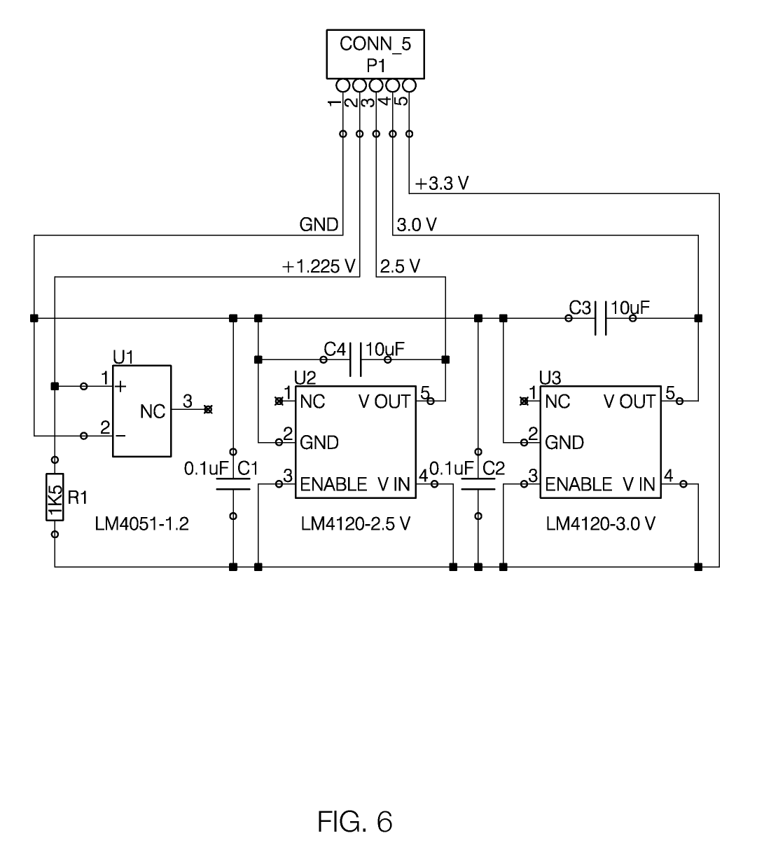

FIG. 6 shows a circuit schematic of the voltage references according to one embodiment. Measurement accuracy depends on the accuracy of the voltage references. They are used to control the thermistor current source and set voltage offset of the amplifier. They are also used to set the full scale of the analog-to-digital converter. Any error in the reference voltages will adversely affect linearity, gain, and range of the data conversion. This application demands high accuracy and therefore external precision voltage references are beneficial. There are many parts available for this task with different degrees of precision, initial accuracy, long term stability, and temperature coefficient. According to one embodiment, part are selected with an error less than 0.05%, and three different voltage levels: 1.225V (LM4051—1.2), for the thermistor constant current source, 2.50V (LM4120—2.5), for the thermistor amplifier offset voltage, and 3.00 V (LM4120—3.0) for setting the full-scale of the analog to digital converter.

According to one embodiment, the CPU of the device is based on the Microchip PIC18F8722 microcontrollers family. The main features of this family are:
High performance 8 bit RISC CPU.
40 MHz/to MIPs sustained operation.
2.0 to 5.5V operation.
Flash, SRAM and EEPROM memory. One of the restrictions in embedded systems is the amount of memory available. This memory shortage is usually overcome by using additional external memories (such as microSD cards).
Digital and analog ports. Digital ports are used to control external LEDs, power the amplifiers and the voltage references. Analog ports are used to acquire thermistor voltage, input ADC voltage references, and monitor batteries level.
4 timers
Up to 25 mA sink/source pin capability. Digital outputs power the amplifiers during sampling and the rest of the time remain in high impedance state to save battery power. This sink/source capability is sufficient for the amplifiers and voltage references.
Up to 16 analog channels. Two analog channels are used for each thermistor, 2 channels as the ADC voltage references, 1 channel to monitor battery level, and another channel to monitor recharging block. Devices with 12 and 16 ADC channels are available in this family. Resolution of the converter is 10 bits, and the measurement range is 10° C. That gives a resolution of 0.01° C. per count, better than the accuracy of the sensors. A Sample and hold circuit is included in the ADC.
Up to 25 Ksps.
Full-duplex asynchronous or half-duplex synchronous addressable USART. Serial interface is employed to communicate with the Bluetooth module.
Programmable low voltage detect.
Watchdog timer.
In-System Programming Method.

According to one embodiment, analog to digital conversion is performed by the converter embedded in the microcontroller. The resolution is 10 bits, with 1023 counts for the maximum level. In this embodiment, there is only one analog-to-digital converter on the microcontroller, namely, only one channel can be converted at a time. An analog multiplexer connects the selected input to the holding capacitor and to the ADC. The input range is set by high and low voltage references. Although VDD or other internal references can be used, the high voltage reference was taken from the precision voltage reference of 3.0 V for more accuracy and stability. The ADC is of the successive approximation type. The steps to perform a conversion are:
Configure pin as analog input.
Select channel.
Enable ADC module.
Start conversion.
Wait for the conversion to be completed.
Read the result.

According to one embodiment, the above tasks are carried out by the firmware, at a sampling rate of one sample per second. Wireline data transmission is very inconvenient in mobile clinical applications. To overcome this limitation, this embodiment includes a Bluetooth interface for wireless data transmission. Additionally, Bluetooth specifications can be supported by a number of different hardware platforms such as cell phones, laptop computers, personal digital assistants and embedded devices. Modules containing the radio hardware are provided by a variety of vendors, and often driver software is also included in order to facilitate design and integration tasks, avoiding, for example, the need for antenna design or RF certification. They virtually eliminate the need to have specific Bluetooth knowledge and simplify programming. The Bluetooth module chosen for this embodiment is a Linkmatik module. It provides a full duplex point-to-point connection similar to that of a standard serial cable, at a transfer rate of up to 50 Kb/s. It can work in slave mode, waiting for a remote device to connect after initialization, master mode, looking for specific devices to connect to, and even connect to other Linkmatik module. We programmed the module to work in slave mode, the host computer connects to the temperature apparatus for off-line or real time data download. Up to 7 devices can be connected in real time simultaneously to the host computer. It is possible to set different security levels, only devices that present the correct password are permitted to connect to the host. The distance communication range is 100 m. It can be powered with a voltage between 3V and 5V. The LinkMatik Bluetooth module is FCC/CE/IC compliant and does not need re-certification if the integral antenna is used. The microcontroller's firmware carries out the following tasks:
Bluetooth module control. A high priority periodical interrupt polls Linkmatik module to read commands from the host computer (set device name, start acquisition, configure mode, get battery level, set date, set time, and retrieve data from memory). It also sends data to the host computer with the format: Date (yyyy-mm-dd), time (hh:mm:ss) and the two temperature samples (float values with two decimal digits). All the fields are separated by tabs. Temperatures are computed according to preprogrammed calibration curves.
Off-line data acquisition in Holter mode. The Bluetooth module is switched off to save power and enable the device to function for up to 10 days. A low priority interrupt samples the two channels every second. The device is in sleep mode for 90% of the time. It is awaken 10 ms before ADC inputs are sampled to get stable values. When 60 samples have been acquired, the mean is computed and stored in EEPROM with the format described above. All data are downloaded to the host computer afterwards, once the recording ends by restarting the device.
Real time data acquisition. The functionality is similar to that of Holter mode, except that Bluetooth module is not switched off, and when the mean is computed, the resulting data are both stored in memory and transmitted to the host computer. Storing data in memory in real time mode enables the device to download lost data in case communication between host computer and temperature apparatus is interrupted (for example, because of device out of range due to monitored patient displacement).
Power control. In order to save battery power, peripherals are switched off during most of the time. They are only active when a temperature sample is acquired. Additionally, microcontroller is in sleep mode until watchdog timer periodically throws an interrupt to awake it. Then, one digital output is set active to power the thermistor amplifier and the voltage references. Both temperature channels are then measured, peripherals are switched off, and microcontroller enters sleep mode again until next cycle. In off-line mode, Bluetooth module is also disabled.

An areal time clock (RTC) based on an internal temporization of 1 s and a low priority interrupt is implemented in firmware in one embodiment. Current date and time are set every time a new recording is started, either in real time or off-line. It provides second, minute, hour, day, month and year information that are associated to each pair of temperature values.

According to one embodiment, the device dimensions are 90×63×28 mm and the weight is 250 g—most of it due to the two AA batteries. The maximum current consumption is 60 mA during Bluetooth discovery and connection. In real time mode, current consumption is around 40 mA, whereas it is lower than 4 mA in Holter mode. To protect the device against X-Rays, the electronic parts are covered by a special fabric. The fabric is a jacquard one, of 210 g/m₂. It is made out of 100% cotton. The fabric is chemically bleached. Some metals have been used with polyurethane for coating it on one face. Afterwards the fabric is dried with a dryer machine. For accurate temperature measurements, it is possible to calibrate the device with high precision resistors. We chose the following reference resistance points within the temperature measurement interval: 1K21, 1K24, 1K3, 1K37, 1K43, 1K54, 1K65, and 1K74 (0.1% tolerance). Calibration points are set for each channel at the host computer, and they are permanently stored in the device.

### C. Analysis System and Analysis Methods According to an Embodiment.

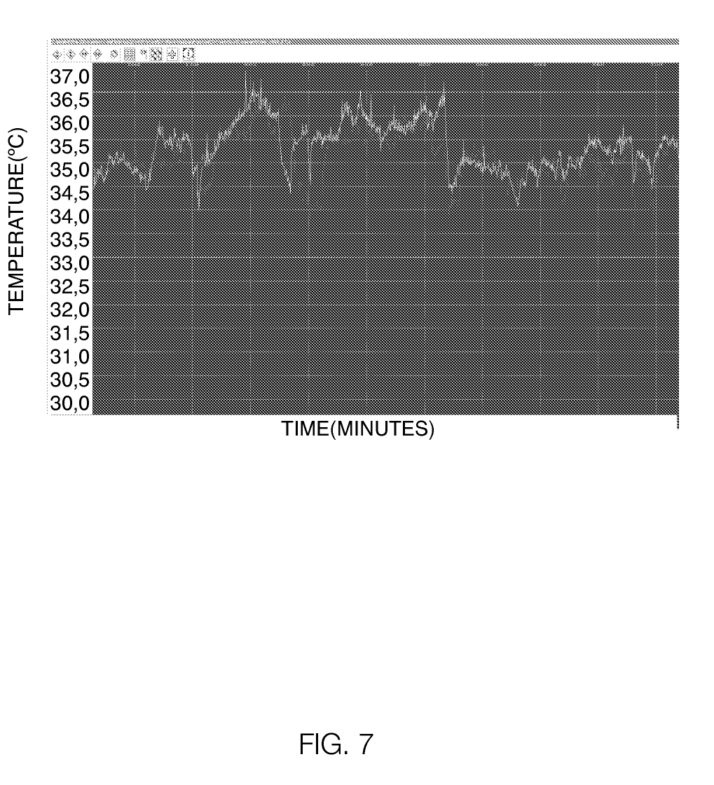

According to one embodiment the temperature apparatus includes an analysis application implemented in a digital computer with one or more processors. This application enables the user to configure a temperature apparatus, start a recording, off-line data download, or calibrate the device. Additionally, it contains standard time-series analysis capabilities and the ability to compute the Approximate Entropy, Lempel-Ziv complexity, Detrended Fluctuation analysis, and Sample Entropy of the temperature recordings. These signal complexity calculations contribute to capture the dynamic aspects of temperature regulation. FIG. 7 displays a screenshot of the software application.

While particular embodiments have been described, it is understood that after learning the teachings included in this description modifications and generalizations will be apparent to those skilled in the art without departing from the spirit of the disclosed embodiments.

### D. Testing, Operation, and Characterization of a Disclosed Embodiment.

The embodiment described above was tested and characterized by conducting a repeatability and reproducibility analysis (R&R) to validate the performance of the proposed device. The objective of this type of analysis is to determine the proportion of measurement variability that is due to the items being measured, the operator, and errors in the measurements over several trials. In an optimal case, all or most of the variability in measurements should be due to the part-to-part variation, not due to operator reproducibility for trial-to-trial repeatability. Reproducibility is the variation obtained among measurements performed by a number of operators, laboratories, or under different conditions, when the item and the measurement device are the same. It quantifies the device sensitivity to its interactions with the environment. In contrast to repeatability, this type of variation can be avoided by proper normalization of the measurement process. Repeatability is the variation obtained when independent measurements of the same sample are taken using the same method, same parts, same device, same operator, and at short time intervals to avoid environment changes. It is

TABLE 1
Results of the ANOVA study conducted to determine the
measurement errors and data variability sources

| Source | SS | DF | MS | F-Ratio | P-Value |
|---|---|---|---|---|---|
| Operator | 0.373986 | 1 | 0.373986 | | |
| Resistors | 13.0283 | 9 | 1.44758 | | |
| Operator * Resistors | 0.44701 | 9 | 0.0496678 | 0.77 | 0.6483 |
| Residue | 2.59587 | 40 | 0.0648967 | | |
| Total | 16.4451 | 59 | | | |

related to intrinsic variability that is not possible to avoid when using a measurement device. In this type of temperature monitor both channels should provide identical high-accuracy temperature readings. We used the calibration precision resistors as the items measured in this test. 10 equal resistors were measured randomly 3 times. ANOVA was used to obtain the measurement errors and data variability sources. The ANOVA Table 1 shows these results. Only the worst case, interaction, was analyzed.

TABLE 2
Characterization of One Particular Embodiment Specifications

| Specification | Result |
|---|---|
| Inputs | 2 channel thermistor |
| Temperature range | 19° C. to 41° C. |
| Characterizations | Steinhart-Hart, YSI-400 |
| Temperature accuracy | ±0.2° C. |
| Temperature resolution | 0.01° C. |
| Measurement interval | 1 minute |
| Data logging | Up to 10 days |
| Averaging | 60 samples/minute |
| Probe connection | Universal 2-way male connector |
| Communications | Bluetooth interface |
| Battery | 2xNiMH AA rechargeable, 500 cycles |
| Size | 90 × 63 × 28 mm |
| Weight | 250 g |

The quantitative results of this R&R analysis confirmed that both channels provide equal and highly accurate measurements, and that measurements are repeatable. Resistors (sensors) contribute to variability in a 75.47%. Reproducibility only adds a 3.5%, which means that both channels are measured equally. Repeatability contributes by a 21.02%, namely, sensors measure with high accuracy. Interaction is not significant. We can conclude that measurements taken with this device are very stable and accurate, since sensors are the factor that accounts for most of the variability.

Additionally, a characterization study designed to document and test each of the temperature apparatus specifications in this embodiment was conducted. The results of this test are shown in Table. 2.

The invention claimed is:

1. A system for taking temperature measurements and analyzing said measurements, comprising:
(a) a wireless temperature acquisition and logging device for acquiring, storing, and wirelessly transmitting said temperature measurements; and
(b) an analysis system implemented in a digital computer with one or more processors in order to analyze and characterize said temperature using one or more time-series analysis methods.

2. The system of claim 1, wherein said wireless temperature acquisition and logging device is especially designed for multi-day, high-frequency, and high-resolution temperature measurements.

3. The system of claim 2, wherein said wireless temperature acquisition and logging device comprises one or more temperature sensors, one or more signal conditioners, one or more signal amplifiers, an analog-to-digital converter, a microcontroller, a memory, a wireless communication module, a power circuit, and a power source.

4. The system of claim 3, wherein said memory is substantially equivalent to a flash memory, said wireless communication module is substantially equivalent to Bluetooth, and said power circuit includes a dc-to-dc converter.

5. The system of claim 2, wherein said time-series analysis methods include complexity analysis techniques.

6. The system of claim 5, wherein said complexity analysis techniques include calculating an Approximate Entropy metric of said temperature.

7. The system of claim 5, wherein said complexity analysis techniques include calculating a Sample Entropy metric of said temperature.

8. The system of claim 5, wherein said complexity analysis techniques include calculating a Lempel-Ziv metric of said temperature.

9. The system of claim 5, wherein said complexity analysis techniques include calculating a Detrended Fluctuation metric of said temperature.

10. A system for taking temperature measurements, analyzing said measurements, and characterizing a patient's temperature time-series to help make clinical decisions regarding a patient's clinical condition, comprising:
(a) a wireless temperature acquisition and logging device especially designed for multi-day, high-frequency, and high-resolution temperature measurements for acquiring, storing, and wirelessly transmitting said temperature measurements; and
(b) an analysis system implemented in a digital computer with one or more processors in order to analyze and characterize said temperature using one or more complexity analysis methods and generate an output temperature complexity metric, said output temperature complexity metric is automatically compared against one or more normality thresholds to help make said clinical decisions regarding said patient's condition.

11. The system of claim 10, wherein said complexity analysis techniques include calculating an Approximate Entropy metric of said temperature.

12. The system of claim 10, wherein said complexity analysis techniques include calculating a Sample Entropy metric of said temperature.

13. The system of claim 10, wherein said complexity analysis techniques include calculating a Lempel-Ziv metric of said temperature.

14. The system of claim 10, wherein said complexity analysis techniques include calculating a Detrended Fluctuation metric of said temperature.

15. A method for assessing a patient's condition, comprising:
(a) collecting a temperature time-series using a temperature acquisition and logging device especially designed for multi-day, high-frequency, and high-resolution temperature measurements;
(b) automatically calculating and displaying a complexity metric at user-specified time intervals and said complexity metric is calculated using a user-specified window length; and
(c) assessing said patient's condition by comparing said complexity metric against one or more normality thresholds known to be indicative of a particular clinical condition.

16. The system of claim 15, wherein said complexity metric includes an Approximate Entropy metric of said temperature.

17. The system of claim 15, wherein said complexity metric includes a Sample Entropy metric of said temperature.

18. The system of claim 15, wherein said complexity metric includes a Lempel-Ziv metric of said temperature.

19. The system of claim 15, wherein said complexity metric includes Detrended Fluctuation metric of said temperature.
